(19) United States
(12) Patent Application Publication
(10) Pub. No.: US 2020/0101310 A1
(43) Pub. Date: Apr. 2, 2020

(54) SYSTEM AND METHOD OF PHOTODYNAMIC SKIN THERAPY

(71) Applicant: Galactic Skin Care, LLC, New York, NY (US)

(72) Inventors: Ellen Marmur, New York, NY (US); Alison Cutlan, Brooklyn, NY (US); Edward Gilchrest, Oxford, CT (US); Nam Thanh Le, New York, NY (US); Matt Prancuk, Brookfield, CT (US)

(21) Appl. No.: 16/617,724

(22) PCT Filed: Apr. 30, 2018
(86) PCT No.: PCT/US18/30160

§ 371 Date: Nov. 27, 2019

(51) Int. Cl. A61N 5/06

(52) U.S. Cl. CPC A61N 5/062; A61N 5/0616; A61N 2005/0652; A61N 2005/0644; A61N 2005/0663; A61N 2005/0626

(57) ABSTRACT

The present disclosure is directed to compositions, devices, a system, and related methods for treatment of skin. The subject matter of the present disclosure also provides maintenance treatment, acne treatment, rejuvenation treatment, and anti-aging treatment for skin. In accordance with some aspects, a photodynamic skin therapy system may generally comprise an apparatus supporting light emissive panels which may in some situations be configured and operative to be coupled to a remote or external device. The light emissive panels may emit light of a selected wavelength in accordance with instructions defining a skin treatment regimen.

### CROSS-REFERENCE TO RELATED APPLICATION

This application claims the benefit of Patent Cooperation Treaty (PCT) patent application number PCT/US18/30160, filed Apr. 30, 2018, the disclosure of which is hereby incorporated herein by reference in its entirety.

### BACKGROUND

Natural products are essential sources of cosmetics and medicines. The World Health Organization estimates that 80% of the world's population relies on traditional medicines made from natural products. The modern pharmaceutical industry is also dependent on plant-based medicines, with as much as 50% of all drugs based on or derived from natural products. As such, plants and other natural products offer excellent sources of health-promoting cosmetics and medicines.

### SUMMARY

The present disclosure is directed to compositions, devices and related methods for treatment of skin. The compositions, devices and related methods of the present disclosure also provide maintenance treatment, acne treatment, rejuvenation treatment, and anti-aging treatment for skin.

In one aspect, the present disclosure provides topical compositions for applying to skin comprising a light activated enzymatic extract; Baobab (Adansonia digitata) seed extract; wild indigo (Tephrosia purpurea) extract; pre- and/or pro-biotic extracts; and a carrier. The compositions may be in a topical gel form.

In some embodiments, the light activated enzymatic extract comprises an enzyme from a blue-green algae (e.g., Anacystis nidulans), such as, for example, photolyase. The light activated enzymatic extract may comprise components selected from the group consisting of photolyase, planktonic enzymes, acai polyphenols, tara tree extract, seaweed extract, and/or combinations thereof.

In some embodiments, the light activated enzymatic extract is activated by light of wavelength from about 420 nm to about 460 nm. In other embodiments, the light activated enzymatic extract is activated by light of wavelength from about 630n to about 700 nm.

In some embodiments, the Baobab seed extract comprises plant ribonucleic acid molecules.

In some embodiments, the wild indigo extract is from the seed of the plant.

In some embodiments, the compositions may further include one or more of the following: willowherb extract, colloidal sulfur, niacinamide, a Thermus thermophilus extract, narnginen, provitamin B5, arnica extract, one or more growth factors, night blooming Chinese cucumber extract, night blooming cereus cactus, chamomile, lavender, and night blooming jasmine.

In another aspect, the present disclosure provides topical compositions for applying to skin comprising a heat activated enzymatic extract, such as, e.g., Thermus thermophiles extract; Baobab (Adansonia digitata) seed extract; wild indigo (Tephrosia purpurea) extract; pre- and/or pro-biotic extracts; and a carrier. These compositions may also be in a topical gel form. These compositions may also comprise light activated enzymatic extracts, as described herein.

In another aspect, the present disclosure provides cosmetic masks for application to skin, the masks comprising a hydrogel or cotton fiber or bio-cellulose matrix comprising the topical composition of the present disclosure embedded therein.

In another aspect, the present disclosure provides methods of treating skin for acne or skin rejuvenation, the methods comprising placing the cosmetic mask described herein on a subject's skin. In some embodiments, the methods may further include exposing the mask to light of a wavelength of about 630 nm to about 660 nm, light of a wavelength of about 420 nm to about 460 nm, or a combination of light sources of a wavelength of about 630 nm to about 660 nm and about 420 nm to about 460 nm.

Embodiments of the present disclosure may also provide adaptogenic action in a mask that encourages balance, epigenetic homeostasis and resistance in skin that is affected by stress, inflammation and imbalance.

The compositions, devices and related methods herein described can be used in connection with pharmaceutical, medical, and veterinary applications, as well as fundamental scientific research and methodologies, as would be identifiable by a skilled person upon reading of the present disclosure. These and other objects, features and advantages of the present invention will become clearer when the drawings as well as the detailed description are taken into consideration.

In accordance with one embodiment, for example, a system of photodynamic skin therapy may generally comprise: an apparatus comprising a housing operative to support a light emissive panel; a user interface operative to receive parameters associated with the skin therapy; regimen control software operative to provide instructions related to the parameters; and control electronics operative to cause the light emissive panel to emit light of a selected wavelength in accordance with the instructions. In some systems, the apparatus may further comprise a memory storing data associated with the skin therapy, wherein the regimen control software accesses the data to provide the instructions. Additionally or alternatively, the apparatus may further comprise a communications interface, wherein the regimen control software receives the parameters via the communications interface.

In some embodiments, a system may further comprise a remote device coupled to the apparatus via the communications interface, wherein the remote device is operative to execute an application program to provide the parameters to the regimen control software. The remote device may be a wireless telephone, and the user interface may allow interaction with the application program executing on the wireless telephone.

In accordance with some systems, the light has a wavelength from about 420 nm to about 460 nm, or from about 630 nm to about 700 nm, or both. As set forth in detail below, the light is operative to activate a photodynamic compound. In some embodiments of a system, the apparatus further comprises a sensor operative to obtain effect data from an area of skin illuminated by the light.

In yet another aspect of the disclosed subject matter, a system of photodynamic skin therapy may generally comprise: a hydrogel matrix for application to an area of skin; a topical composition comprising an enzymatic extract embedded within the hydrogel matrix; an apparatus comprising a housing operative to support a light emissive panel; a user interface operative to receive parameters associated with the skin therapy; regimen control software operative to provide instructions related to the parameters; and control electronics operative to cause the light emissive panel to emit light of a selected wavelength in accordance with the instructions; wherein a portion of the hydrogel matrix is selectively illuminated by the light.

In some such systems, the apparatus may further comprise a sensor operative to obtain effect data from an area of the hydrogel matrix illuminated by the light. In accordance with one aspect, the regimen control software selectively modifies the instructions responsive to the effect data.

In some systems, the topical composition further comprises at least one of Baobab seed extract, wild indigo (Tephrosia purpurea) extract, pre- and/or pro-biotic extracts, and a carrier. The enzymatic extract may be a photodynamic compound, a thermodynamic compound, or both.

As set forth in detail below, in one embodiment, a method of photodynamic skin therapy may generally comprise: embedding a topical composition within a hydrogel matrix, the topical composition comprising an enzymatic extract; applying the hydrogel matrix to an area of skin; and selectively illuminating a portion of the hydrogel matrix in accordance with a skin treatment regimen and the topical composition; wherein the selectively illuminating comprises: providing an apparatus comprising a housing operative to support a light emissive panel; receiving parameters associated with the skin treatment regimen via a user interface; instantiating regimen control software operative to provide instructions related to the parameters; and employing control electronics operative to cause the light emissive panel to emit light of a selected wavelength in accordance with the instructions. In some such methods, the selectively illuminating further comprises employing a sensor operative to obtain effect data from the portion of the hydrogel matrix and selectively modifying the instructions responsive to the effect data.

In accordance with one method, the topical composition may further comprise at least one of Baobab seed extract, wild indigo (Tephrosia purpurea) extract, pre- and/or pro-biotic extracts, and a carrier. Additionally or alternatively, the enzymatic extract is a thermodynamic compound.

### BRIEF DESCRIPTION OF THE DRAWINGS

The foregoing and other aspects of various embodiments of the disclosed subject matter will be apparent through examination of the following detailed description thereof in conjunction with the accompanying drawing figures. It is to be noted that the drawing figures show particular variants, but the present disclosure is not, however, restricted to the embodiments illustrated. The same components in the drawing figures are given the same reference numerals unless otherwise indicated. In the drawings figures:

FIGS. 1-8 and 24 are perspective views of embodiments of a system in accordance with the present disclosure, illustrating respective display configurations;

FIGS. 9 and 10 are simplified representations of light emission strategies in accordance with aspects of the present disclosure;

FIG. 11 illustrates different embodiments of a mechanical attachment in accordance with an aspect of the present disclosure;

FIGS. 12A-12E illustrate different user interface mechanisms facilitating operation of a system in accordance with the present disclosure;

FIG. 13 is a simplified block diagram illustrating functional blocks of an illumination apparatus in accordance with one embodiment;

FIG. 14 is a simplified flow diagram illustrating general operational flow of one embodiment of a method in accordance with the present disclosure; and

FIGS. 15-23 illustrate different user interface screens facilitating interaction with a system in accordance with some embodiments.

### DETAILED DESCRIPTION

Several aspects of the disclosure are described below. It should be understood that numerous specific details, relationships, and methods are set forth to provide a full understanding of the disclosure. One having ordinary skill in the relevant art, however, will readily recognize that the aspects of the disclosure can be practiced without one or more of the specific details or practiced with other methods, protocols, and animals. The present disclosure is not limited by the illustrated ordering of acts or events, as some acts may occur in different orders and/or concurrently with other acts or events. Furthermore, not all illustrated acts, steps or events are required to implement a methodology in accordance with the present disclosure. Many of the techniques and procedures described, or referenced herein, are well understood and commonly employed using conventional methodology by those skilled in the art.

Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular embodiments only and is not intended to be limiting of the invention. As used herein, the indefinite articles “a” and “an,” and the definite article “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the term “comprising.”

As used herein, the term “subject” refers to an animal. Typically, the terms “subject” and “patient” may be used interchangeably herein in reference to a subject. As such, a “subject” includes a human that is being treated for a skin ailment, such as acne, or for anti-aging or skin rejuvenation.

The term “animal,” includes, but is not limited to, dog, cat, pig, monkey, chimpanzee, and human.

The present disclosure provides compositions, devices and related methods of treatment for skin, such as for skin rejuvenation, acne treatment or anti-aging (e.g., wrinkle reduction). In one aspect, the present disclosure provides topical compositions for applying to skin, the compositions comprising a light activated enzymatic extract; Baobab seed extract; wild indigo (Tephrosia purpurea) extract; pre- and/or pro-biotic extracts; and a carrier.

The term “carrier” refers to a diluent, adjuvant, hydrocolloid, emulsifying agent, excipient, and/or vehicle with which the composition is administered. In preferred embodiments, the carrier is utilized as a topical gel or serum. Such pharmaceutical carriers can be sterile liquids, such as water and oils, including those of petroleum, animal, vegetable or synthetic origin, such as plant seed oils, nut oils, mineral oil, lanolin, esters, wax based ingredients, and the like. Suitable pharmaceutical excipients include starch, glucose, sucrose, gelatin, lactose, malt, rice, flour, chalk, silica gel, sodium stearate, glycerol monostearate, talc, sodium chloride, glycerol, propylene glycol, butylene glycol, propanediol, sorbitol, water, ethanol and the like. The composition(s) may also contain wetting or emulsifying agents or suspending/diluting agents, or pH buffering agents, or agents for modifying or maintaining the rate of release of the compositions, such as but not limited to, sodium acrylate, acryloyldimethyl taurate copolymer, isohexadecane, and polysorbates, carbomers, ceteareth-20 (non-ionic polyoxyethylene ether of higher saturated fatty alcohols (cetyl/stearyl alcohol), thickening alcohol cetearyl alcohol NF, and combinations thereof, at total concentrations of, for example, about 0.5 to 5% (v/v). The compositions can take the form of solutions, suspensions, emulsions, gels, creams, sustained-release compositions and the like. Compositions can include standard carriers such as pharmaceutical grades of mannitol, lactose, sodium saccharine, starch, magnesium stearate, cellulose, magnesium carbonate, etc. Such compositions will contain an effective amount of the components of the compositions together with a suitable amount of carrier so as to provide the proper form to the subject based on the mode of administration to be used. The compositions can also comprise one or more preservatives, astringents, humectants, and anti-oxidants, used either individually or in a mixture thereof. Antioxidants can include, but are not limited to, Leontopodium alpinum extract (bisabolane, sitosterol, tannin, chlorogenic acid, apigenin-7-glucoside, luteolin, luteolin-4-glucoside); Coenzyme Q10 (with vitamin E & C), (2,6-di-tert-butyl-4-methylphenol), any FDA-approved antioxidant widely used as stabilizer, citric acid, vitamin A (retinol palmitate), and combinations thereof. Preservatives can include, but are not limited to, tea tree essential oil (for example, at a concentration of 0.01 to 3.0% w/w of the composition), rosemary leaf extract, phenylpropenes, cineole (eucalyptol), neem oil, propolis (produced by bees), rosemary extract, citric acid, alpha tocopherol (vitamin-E), potassium sorbate, sodium benzoate and combinations thereof.

The compositions can also comprise solid lipid emollient ingredients, such as USP petrolatum, solid lipid nanoparticles (SLN) or microparticles, nanostructured or microstructured lipid carriers, bees wax, jojoba gel, carnauba wax, octyldodecanol, tryglyceride, and combinations thereof. As would be understood by those skilled in the art, nanoparticles include particles that are about 100 nm or less and microparticles include particles about 200 nm or greater in size.

In accordance with one implementation, the light activated enzymatic extract comprises an enzyme from a blue-green algae. The light activated enzymatic extract may include components such as photolyase, other planktonic enzymes, acai polyphenols (red light activated), wolfberry (goji) amino acids (red light activated), tara tree extract, seaweed extract, and combinations thereof.

In some embodiments, the enzyme is photolyase. Photolyase absorbs visible light to directly cleave and reverse damage caused by shorter wavelength UV. The photolyase may be derived from a photosynthetic plankton called Anacystis nidulans. In additional embodiments, the photolyase enzyme may be contained in multilamellar liposomes, which may be about 200 nanometers in size. The liposomes can be formed from pure soy phospholipids.

In some embodiments, the light activated enzymatic extract may be activated by light of wavelength in a range from about 420 nm to about 460 nm. This wavelength of light is visually in the blue spectrum. In other embodiments, the light activated enzymatic extract may be activated by light of wavelength in a range from about 630 nm to about 700 nm. This wavelength of light is visually in the red spectrum. In yet other embodiments, the light activated enzymatic extract may be activated by either light of wavelength from about 420 nm to about 460 nm or light of wavelength from about 630 nm to about 700 nm.

The compositions described in this disclosure can include pre/probiotics which can include alpha-glucooligosaccharides, plant juices rich in beta-fructooligosaccharides (from jicama or yacon tubers (Polymnia sonchifolia), and Lactobacillus probiotic bacteria (L. casei, L. acidophilus). The pre/probiotics act to stimulate and rebalance the beneficial microflora on the skin, increasing natural skin defenses. They also protect the skin from an overgrowth of acne causing bacteria.

In some embodiments, the composition described herein includes a Baobab seed extract. Such extracts may comprise plant ribonucleic acid molecules. Baobab seed extract is associated with adaptogenic effects including maintenance of the key enzymes essential for the maturation of skin microRNAs (e.g., Drosha and Dicer) to help improve epigenetic homeostasis and resistance. An increasing number of scientific studies are uncovering the biological activity of small RNAs on body health and wellbeing, in particular the small RNAs found in the food provided by the plants. These small RNAs have been described in plants as being a possible origin for rapid adaption to environmental stress for survival and longevity. The Baobab seed extract is, thus, rich in plant small RNAs to improve and maintain skin homeostasis while helping reduce the appearance of wrinkles and provide skin hydration. Baobab is also shown to increase collagen, elastin and HA synthesis and hydration.

In some embodiments, the composition described herein includes wild indigo (Tephrosia purpurea) extract, which may be from the seed of the plant. Wild indigo is a native Indian plant used in the Ayurvedic tradition for its adaptogenic effects on skin. A specific extraction from the seeds of this plant is utilized to obtain a condensate enriched in specific sugars, including stachyose and ciceritol. The wild indigo extract's adaptogenic effects include its ability to break down cortisol production by skin cells, its ability to activate the release of a natural calming neuropeptide acting on mood, and it improves skin tone. The extract also stimulates the production of beta-endorphin, a natural relaxing and pain-relief peptide, by skin cells and stimulates the expression of genes involved in the skin cells' homeostasis. Genes stimulated include, for example, hem oxygenase, the major cellular response to stress factors; NADPH quinone dehydrogenase 1 and heme oxygenase, involved in oxidative stress response; and genes involved in iron and heavy metal detoxification (Ferritin, metallothionein), antimicrobial markers (LL37 cathelicidin), skin barrier markers (ATP binding cassette transporter), anti-inflammatory markers (interleukin 1-alpha), and redox homeostasis markers (thioredoxin reductase).

The compositions provided in this disclosure can further include one or more of the following: willowherb extract (which is an anti-acne botanical that also soothes and reduces redness), colloidal sulfur (which is a gentle keratolytic with pore cleaning action), niacinamide (improves healthy skin tone and texture), a Thermus thermophilus extract, narnginen (an antioxidant), provitamin B5 (calms post-procedure redness), arnica extract (prevents bruising and trauma), one or more growth factors (such as, for example, TGF-beta), night blooming Chinese cucumber extract (Trichosanthes kirilowii), night blooming cereus cactus (moisturizes, softens and soothes skin), chamomile, lavender, and night blooming jasmine.

Night blooming Chinese cucumber extract (Trichosanthes kirilowii) (generally obtained from the root of the plant) protects skin against DNA damage induced by ultra-violet (UV) light, improves wrinkles in skin, and normalizes any disturbed circadian rhythm of the skin.

Thermus thermophilus is a micro-organism that thrives in deep sea thermal vents. Its extract is activated by heat and has the adaptogenic effects of preventing visible signs of photo-aging (spots, wrinkles, and dryness). It can counteract reactive oxygen species (ROS) production and promotes epidermal integrity and moisture maintenance post procedure. It would be understood that those skilled in the art that the present disclosure encompasses other micro-organismal extracts that are activated by heat.

In another aspect, the present disclosure provides a cosmetic mask for application to skin which contains the compositions described herein embedded in, disposed on, infused or integrated into, or impregnated into, or otherwise applied to a hydrogel or bio-cellulose or fibrous matrix or other suitable substrate. Such masks can be designed to cover the entire face, forehead and neck regions of a subject. In some implementations, a mask may cover the face, neck, shoulders, and décolleté of a subject, although smaller masks may be used for more targeted applications.

In a further aspect, the present disclosure provides a method of treating skin for acne or skin rejuvenation or preventing skin aging comprising placing the mask on a subject's skin. In particular, when treating for acne, the mask described herein can be exposed to blue wavelength light (about 420 nm to about 460 nm) upon application on the face. This kills unwanted surface acne causing bacteria, while also activating the light activated enzymes embedded in the mask.

“Treating” or “treatment” of any skin ailments refers, in one embodiment, to ameliorating the ailment (i.e., arresting or reducing the development of the ailment (i.e., acne) or at least one of the clinical symptoms thereof). In another embodiment, “treating” or “treatment” refers to ameliorating at least one physical parameter, which may not be discernible by the subject. In yet another embodiment, “treating” or “treatment” refers to modulating the ailment, either physically, (e.g., stabilization of a discernible symptom), physiologically, (e.g., stabilization of a physical parameter), or both. In yet another embodiment, “treating” or “treatment” refers to delaying the onset of the ailment (wrinkles (i.e., aging)). It would also be understood by a skilled artisan how to use the compositions and masks described herein for therapeutic purposes without undue experimentation based on the teachings provided throughout the specification.

“Preventing” or “prevention” refers to a reduction or slowing of skin aging symptoms (e.g., wrinkles).

It is also understood and contemplated that aspects of the present disclosure can provide more than two ingredient components in the compositions and masks herein disclosed. For example, a composition can comprise a light activated enzyme and/or any one or more component disclosed herein, such as, e.g., heat activated enzymes. Also, the disclosed methods can comprise the simultaneous or separate administration of multiple components by way of the masks described herein. Thus, the present disclosure may further include the administration of a mask having a third, fourth, etc. ingredient, wherein the third, fourth, etc. ingredient is administered separately (in a separate mask), but at substantially the same time as the other active ingredients, or hours or days after the first administration of ingredients (mask).

Further, the masks have unique and innovative photo-dynamic ingredients that activate their anti-aging powers upon exposure to either red or blue light (as described previously herein). Upon application of the mask, one can also expose the mask to light of a wavelength of about 630 nm to about 660 nm, a light of a wavelength of about 420 nm to about 460 nm, or a combination of light of a wavelength of about 630 nm to about 660 nm and about 420 nm to about 460 nm, to activate any light activated enzymatic extracts in the compositions embedded therein. The use of red wavelength light also stimulates anti-aging action within the skin. The use of such photodynamic therapies is further described in Juhasz et al., “The two faces of fractionated photodynamic therapy: Increasing efficacy with light fractionation or adjuvant use of fractional laser technology” Journal of Drugs in Dermatology, 15:11 (November 2016), which is incorporated herein in its entirety.

The masks described herein may be made of a natural hydrogel or bio-cellulose material, one of the most advanced and highest quality materials available. This unique material contains the compositions described within its matrix and is able to deliver the composition more efficiently and deeper into the skin (almost 200×) than results that can be achieved using the compositions alone (without a mask or fibrous/gel matrix or other material to which the composition may be applied). The masks' thin and pliable texture is like a ‘second skin’ and adheres to the skin effortlessly, with a cooling and soothing feel to it. Unlike other sheet masks, the composition embedded in the matrix makes it luxurious and easy to use, and not messy or wet like standard sheet masks. The masks are designed to treat the entire cosmetic area of the face, the neck, plus décolleté. This larger surface area and custom fit is a unique and defining factor compared to conventional applications. Subsequent references to masks, sera, enzymatic extracts, hydrogels, matrices, or bio-cellulose materials should be understood in the context of the foregoing detailed description.

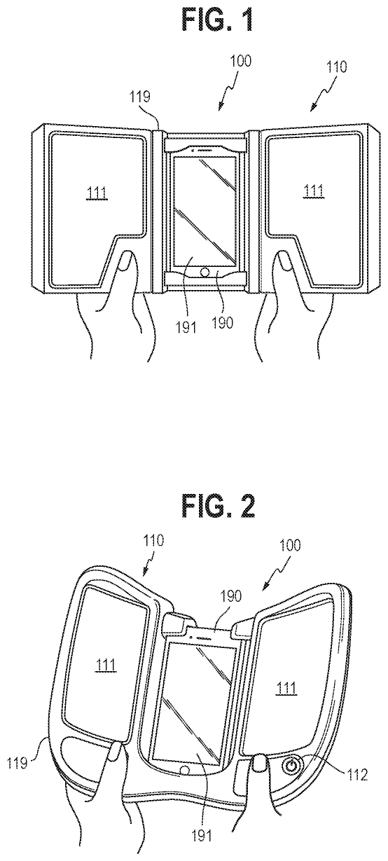

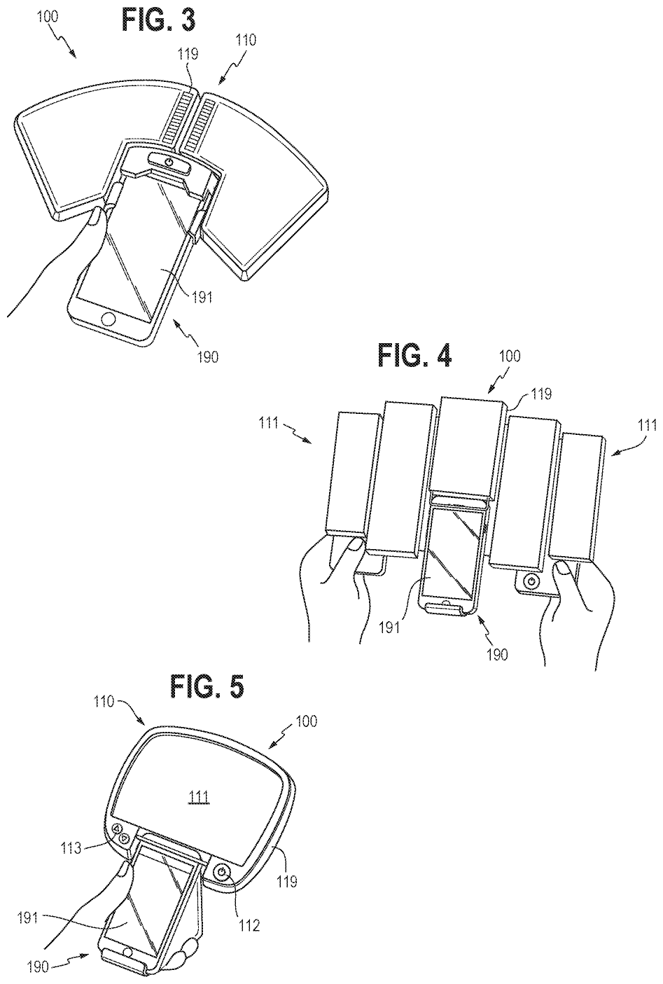

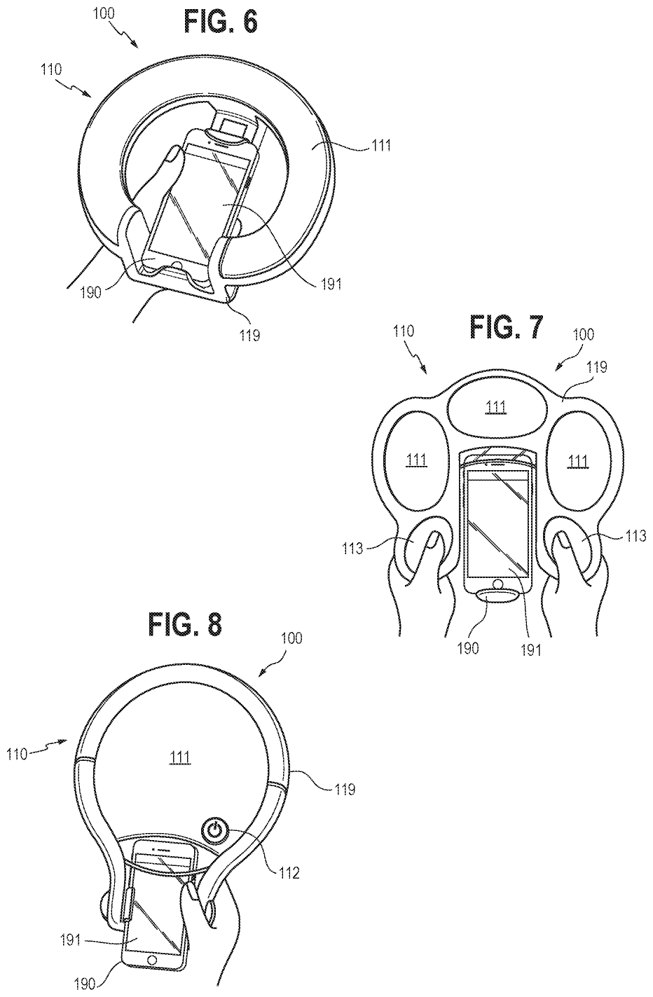

Turning now to the drawing figures, and as noted above, FIGS. 1-8 and 24 are perspective views of embodiments of a system in accordance with the present disclosure, illustrating respective display configurations. As illustrated in the various drawing figures, a photodynamic skin therapy system 100 may generally comprise an illumination apparatus 110 which may in some situations be configured and operative to be coupled to a remote or external device 190. In operation in accordance with some embodiments, apparatus 110 and device 190 cooperate to provide the functionality set forth below.

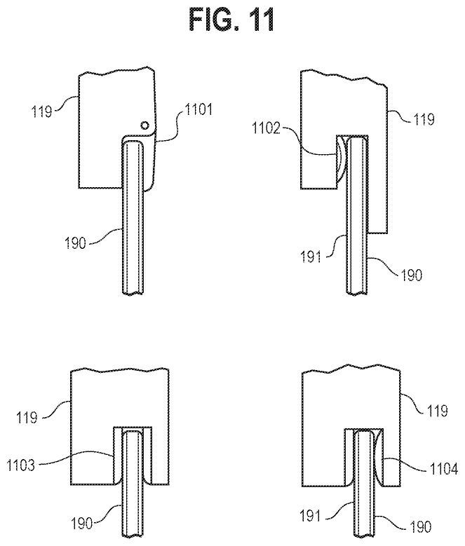

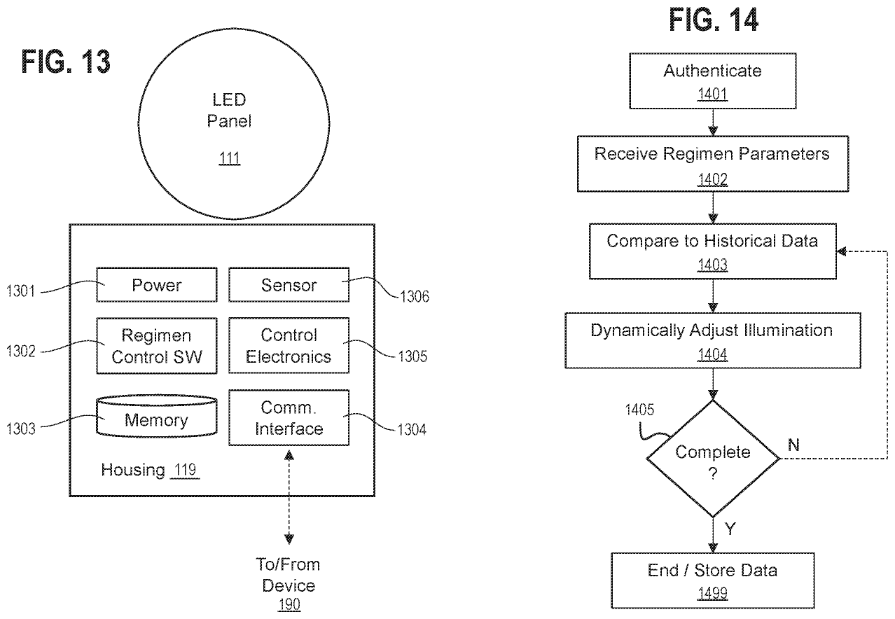

Apparatus 110 generally comprises a housing 119 to support one or more illumination panels 111 and various other electronic, mechanical (such as switches, dials, rocker panels, etc.), and other components as set forth in more detail with specific reference to FIGS. 11 and 13. Housing 119 may be constructed of metal, ceramic, plastics, or other materials generally known in the art of consumer electronics manufacture, and may comprise a variety of such materials. In some implementations, it may be desirable to use injection molding techniques to construct housing 119 of a plastic material; additionally or alternatively, some portions or all of housing 119 may be constructed of a metal such as aluminum, stainless steel, or titanium. Those of skill in the art will appreciate that a metal housing 119 may be more durable than one made of plastic, but that the selection of materials is generally a design choice that may be influenced by factors (such as cost, weight, and constraints associated with manufacturing processes) that are unrelated to the functional aspects of system 100 as set forth below.

Illumination panels 111 may be operative to emit electromagnetic radiation under control of apparatus 110, either independently or in cooperation with or as a function of data sets or instructions received from device 190. In that regard, apparatus 110 (and housing 119, in particular) may include appropriate user input mechanisms such as a power switch or button (illustrated as button 112) or other controls 113 such as, for instance, contrast or intensity controls, biometric data sensors, and other input devices as is generally known in the art. The present disclosure is not intended to be limited by the nature or orientation of power button 112 or controls 113, but it is worth noting that some embodiments of apparatus 110, particularly those capable of operation independent of device 190, may benefit from independent control mechanisms.

In some implementations, panels 111 may be operative to emit light having a wavelength in a range from about 420 nm to about 460 nm. This wavelength of light is visually in the blue spectrum. In other embodiments, panels 111 may be operative to emit light having a wavelength in a range from about 630 nm to about 700 nm. This wavelength of light is visually in the red spectrum. As noted above, the wavelength of light selected for emission by panels 111 may be influenced by, for example, a light activated (i.e., photodynamic) enzymatic extract for use in skin therapy; some such enzymatic extracts may be responsive to wavelengths from about 420 nm to about 460 nm or from about 630 nm to about 700 nm. It will be appreciated that the desired or required output of panels 111 be selected to interact suitably with such an enzymatic extract or as a function of a prescribed or desired skin care regimen independent of any extracts or other applications on the treated area upon which the light is incident. In that regard, it is noted that the present disclosure is not intended to be limited to any particular wavelength of light emitted by panels 111, and that the emissive qualities of panels 111 may be selected in accordance with any number of factors, including therapeutic extracts, manufacturing considerations associated with the technologies employed at panels 111, desired treatment regimens, the number and size of panels 111 implemented at apparatus 110, or a combination of these and other factors. For instance, wavelengths in the green, yellow, white, and purple portions of the visible spectrum may also have utility in certain implementations. Panels 111 may be selected to provide output in a desired wavelength range suitable for the desired skin treatment regimen or expected output requirements.

Panels 111 may comprise or be embodied in liquid crystal display (LCD) panels, light emitting diode (LED) panels, organic LED (OLED) panels, or other display technologies as dictated by design choices and the overall desired operability of system 100. Those of skill in the art will understand that the technology implemented by panels 111 should provide for emission of light in a desired wavelength range and supporting desired output intensity levels, but that the selected display technology should not otherwise be limit by the functionality set forth herein. It is noted that “intensity” in the context of the present disclosure includes “radiant intensity,” “luminous intensity,” “irradiance,” “radiance,” and other metrics generally associated with luminance or “brightness” of light, as those terms are generally understood in the photometry arts.

In some modes of operation, apparatus 110 may cooperate with remote or external device 190, as noted above. In that regard, device 190 may be implemented as a wireless telephone, a person digital assistant (PDA), a tablet computer, or some other electronic device capable of bi-directional data communication with apparatus 110 that may influence output of panels 111, for example, under control of a software application program executing on device 190. In the illustrated embodiments, device 190 is depicted as a telephone having a display 191, though other embodiments are also contemplated. For instance, while a device 190 that is generally small, light-weight, and portable may be coupled to apparatus 110 as indicated in the drawing figures, it may also be desirable to couple apparatus 110 with a larger device such as a laptop computer, a desktop computer, a workstation, or some other electronic or digital computing device; this may be accomplished via suitable cabling, for example, or via a wireless communication protocol such as Wireless Fidelity (WiFi™), Bluetooth™, or some other wireless near-field communications (NFC) technology.

In some embodiments such as those illustrated in FIGS. 1-8 and 24 where device 190 has a display 191 that is situated proximate panels 111, the light emission functionality set forth below may also utilize the functionality of display 191 in cooperation with output of panels 111; this feature may not be possible if apparatus 110 is employed in cooperation with a device 190 such as a desktop computer, for instance. Where such a remote computer has a display situated close enough to apparatus 110 and panels 111, and assuming that the display is capable of providing light in the appropriate wavelength and intensity ranges for a particular treatment protocol, than such a display may be employed in cooperation with panels 111 to provide the light emission strategies described below.

Those of skill in the art will appreciate that the embodiments depicted in FIGS. 1-8 and 24 are representative only, and that additional panels 111 or alternative structural arrangements are contemplated that may be consistent with the operational characteristics set forth herein. The present disclosure is not intended to be limited by the number of panels 111 (or by their orientation or relative positions) incorporated into or embodied by apparatus 110.

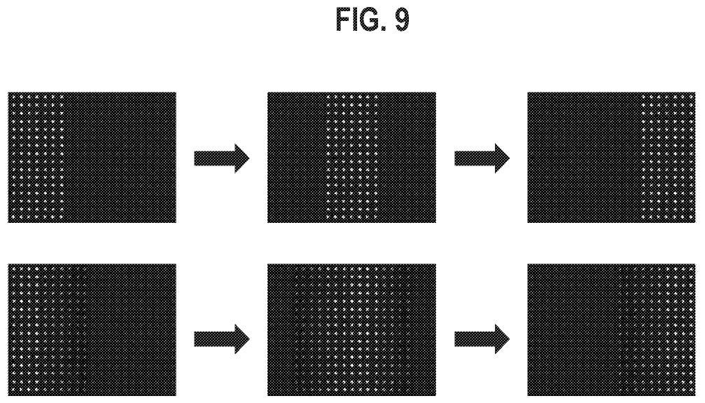

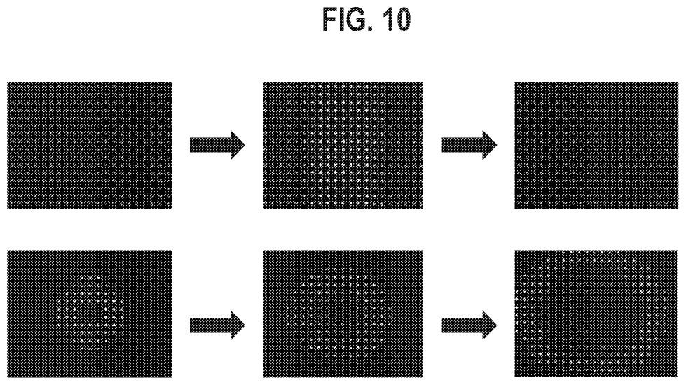

FIGS. 9 and 10 are simplified representations of light emission strategies in accordance with aspects of the present disclosure. These figures illustrate how output of light from panels 111 changes over time in some embodiments; time increases from left to right in FIGS. 9 and 10. Further, it is noted that the changes in light emission over time are illustrated with reference to a single panel 111; in embodiments where multiple panels 111 are employed, however, the multiple panels 111 can be implemented as a single panel system such that the illustrated light propagation is distributed over the entire panel system (as opposed to, for example, each individual panel 111 displaying the illustrated effects independently).

As illustrated in FIG. 9, light emission may propagate across panel 111 from left to right over time (though propagations from right to left or in a vertical direction are also contemplated). The top of FIG. 9 illustrates an embodiment in which there is a sharp contrast between portions of panel 111 (or a combination of panels 111) that are emitting light and portions that are not; the bottom of FIG. 9 illustrates a similar embodiment in which the contrast is softened or faded, such that the intensity of output light is gradually increased and decreased as the output propagates across the panel 111 (or combination of panels 111). Where multiple panels 111 are employed, for instance, the images at the left of FIG. 9 may indicate that the two left-most panels in FIG. 4 are emitting light at a desired intensity, whereas the images in the middle of FIG. 9 may indicate that, at some subsequent time, the middle three panels 111 in FIG. 4 are emitting light (again, at a desired and dynamically changing intensity); similarly, the two images at the right of FIG. 9 may indicate that the two right-most panels 111 in FIG. 4 are active at a point even later in time.

The top portion of FIG. 10 illustrates a pulsing or “on/off” illumination strategy. At a first time period, the panel 111 or combination of panels 111 may not emit light, or may emit light at a low intensity. This may be followed by a burst of light, as indicated in the middle frame; the duration, intensity, and wavelength of the burst may be selectively controlled, as noted above, in accordance with a variety of factors, and may be output by the entire panel 111 or combination of panels 111 or only by a selective portion of the overall panel area. This burst may be followed in time (i.e., in the right-most image) by a period of no light or of light with a relatively lower intensity.

The lower portion of FIG. 10 illustrates a similar strategy that employs radiating circles (though other shapes such as rectangles and ovals are contemplated) of light. As indicated, a desired shape, such as a circle, may begin small, e.g., near the center of panel 111 or combination of panels 111, and grow in size over time until it recedes at the peripheral extremities of the display area; additionally, the wavelength and intensity may also be manipulated as the shape grows.

It is noted that FIGS. 9 and 10 illustrate only a few of the myriad possibilities for fluctuating, pulsing, moving, or otherwise controlling the light that is output from panel 111 or a combination of panels 111. The specific treatment regimen may be selectively controlled via components of apparatus 110 either individually or in cooperation with device 190 as noted above and as set forth in more detail below. Specific wavelengths, intensities, pulse or fade durations, and other regimen-specific output parameters may be under software control, selectively adjustable, manually set by a user, patient, care-giver, or physician, or may otherwise be pre-determined or dynamically adjustable in accordance with desired overall operational characteristics of system 100. Wavelength and output intensity and duration may be selected, for instance, as a function of an enzymatic extract applied to an area to be illuminated, or depending upon a goal of the treatment (for instance, light in the blue range of the visible spectrum may provide a soothing or calming effect, whereas light in another wavelength range may be invigorating or rejuvenating). The present disclosure is not intended to be limited by any particular wavelength, intensity, pulsing or fading effect, or other characteristic of the light emission strategies facilitated by panels 111.

FIG. 11 illustrates different embodiments of a mechanical attachment in accordance with an aspect of the present disclosure. As noted above with reference to FIGS. 1-8 and 24, it may be desirable in some circumstances that device 190 be coupled to apparatus 110 as illustrated. FIG. 11 depicts some mechanisms that may physically accomplish such a coupling; in each case, housing 119 of apparatus 110 may employ a physical structure to engage device 190 securely during operation of system 100. At the top left of FIG. 11, housing 119 includes an external clip 1101 to engage device 190. At the top right of FIG. 11, housing 119 includes a front internal clip 1102 to engage device 190 (in this context, “front” refers to the side of device 190 that includes display 191). At the bottom left of FIG. 11, housing 119 includes a two-sided clamp 1103 to engage device 190. Finally, at the bottom right of FIG. 11, housing 119 includes a back internal clip 1104 to engage device 190.

It will be appreciated that the mechanical attachments 1101-1104 illustrated in FIG. 11 are generally known in the art, and are only representative of some of the various mechanisms that may facilitate coupling of device 190 to apparatus 110 (and, in particular, to housing 119). In some embodiments, mechanical attachments 1101-1104 may additionally comprise suitable electronic connections, power connections, or both, that are operative physically to couple components of device 190 to cooperating or counterpart components of apparatus 110 (for example, to enable bi-directional data communications and to allow operating power to flow freely between device 190 and apparatus 110). For instance, mechanical attachments 1101-1104 may comprise a universal serial bus (USB) or Thunderbolt™ connector, or some other electronic connector generally known in the art or developed in accordance with known principals; these connectors are not illustrated in FIG. 11 for clarity, but those of skill in the art will appreciate that they may be readily implemented in such a manner as to enable a physical connection between electronic or other operative components of device 190 and apparatus 110 when mechanical attachments 1101-1104 are engaged to attach device 190 to housing 119. In these situations, the type and location of such a connector may be influenced by the type of device 190 sought to be coupled to apparatus 110, the physical structure of mechanical attachments 1101-1104, and the type of coupling (power, data, or both) intended to be effectuated.

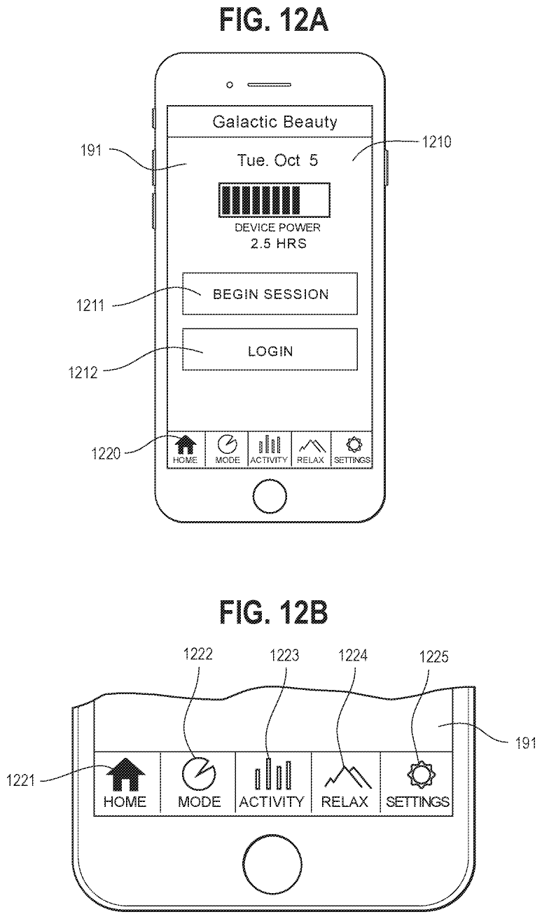

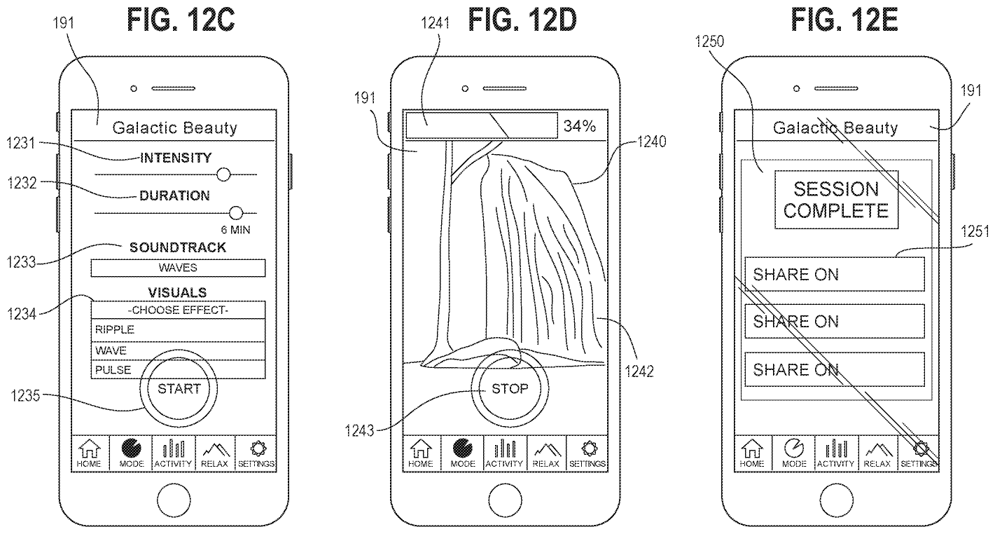

FIGS. 12A-12E illustrate different user interface mechanisms facilitating operation of a system in accordance with the present disclosure. In the following discussion, it is assumed that the user interface mechanisms and elements are displayed on a display 191 associated with device 190, and that device 190 is executing a software application program that is operating in cooperation with apparatus 110. As noted above, however, apparatus 110 may also operate independent of device 190 in some embodiments, and so it is contemplated that the user interface elements may be displayed on one or more panels 111 associated with apparatus 110 (particularly where apparatus 110 or housing 119 includes associated buttons or controls 112 and 113 described above).

FIG. 12A depicts a start or “home” screen 1210, which may be displayed when a user first initializes a software application or other instruction set configured and operative to provide the functionality described herein. In that regard, an application or other executable program may be installed on apparatus 110, device 190, or both that controls output of panels 111 in accordance with a pre-determined or dynamically adjustable protocol or “treatment regimen.” Upon instantiation or execution of such an application program, home screen 1210 may offer up an option to begin a session or regimen (using button 1211), for instance, or to sign in (using button 1212). In some embodiments, for example, it may be desirable to allow the application program to store and maintain records associated with a user or with multiple users, such that treatment histories, preferences, and prescribed, desired, or favorite treatment regimens may be associated with particular users for easy recall, to monitor progress, and the like. In such instances, a user profile or other user-specific data storage may be created and maintained by the application program; the act of “signing in” (such as with button 1212) may enable an application program having control or influencing operation of system 100 to identify the particular user interacting with apparatus 110, device 190, or both and to present customized user interface mechanisms that are particular to that particular user.

It is also noted that the term “button” is intended to be interpreted as a generic mechanism for interacting with the user interface. Those of skill in the art will appreciate that other alternatives include sliders, radio buttons, check boxes, tabs, or other icons. Typically, display 191 associated with device 190 is a touch screen or touch-sensitive display, such that tapping a region of display 191 containing a button or other icon (such as buttons 1211 and 1212) effectuates an action to be taken by the application program. The present disclosure is not intended to be limited by any particular user interface mechanism or the by the terminology used to describe such mechanisms generally known in the art.

Home screen 1210 may also include a menu bar 1220 or other administration area, a more detailed view of which is illustrated in FIG. 12B. Menu bar 1220 may include a home button 1221 (operative to return the user interface to the home screen), a mode button 1222 (operative to influence an operating mode of the application program), an activity button 1223 (operative to cause the user interface to display a user's recent or historic use activity), a relax button 1224 (operative to initiate a particular treatment regimen or relaxation routine stored by the application program), and a settings button 1225 (operative to cause the application program to display settings or other operational parameters that may be viewed or changed as desired). These types of features are generally known in the art and employed by various application programs to enable a user to interact with the program as desired and to manipulate particular settings or operational characteristics of the application program.

FIG. 12C depicts a mode selection screen 1230 in which intensity (1231) of the light, duration (1232) of the regimen, audio selection or soundtrack (1233), and visual stimuli (1234) for display on display 191 during the regimen may be selected. A treatment regimen that proceeds in accordance with the selected parameters may be started, for example, by selecting a start button 1235 or similar user interface mechanism. It is noted that the mode selection screen 1230 is only representative. For instance, in situations where display 191 of device 190 is used as part of a combination of panels 111 to emit light at selected wavelengths and intensities, selection of visual stimuli (such as at button 1234) may not be offered. Additionally or alternatively, soundtracks (1233) and visuals (1234) may be selected automatically by the application program, for example, as a function of the intensity (1231) and duration (1232) parameters that have been set or selected.

FIG. 12D depicts a user interface that may be displayed during ongoing operation in relax mode (such as may be initiated by selection of relax button 1224). Relax screen 1240 may include a timer or progress bar 1241 and depict a relaxing or soothing image 1242 (as noted above, where display 191 is in use for light emission, this image 1242 may be omitted or displayed only intermittently). A stop button 1243 or other user interface feature may be used to exit relax mode, for example, before progress bar 1241 times out.

FIG. 12E depicts a session complete screen 1250 to be displayed when a treatment regimen has concluded. The software application may display session complete screen 1250 automatically upon expiration or termination of the most recent regimen, and may include buttons 1251 to allow a user to share progress on social media networks, for example, or to store details of the most recent session for later retrieval, review, or further processing (such as for example, as historical data).

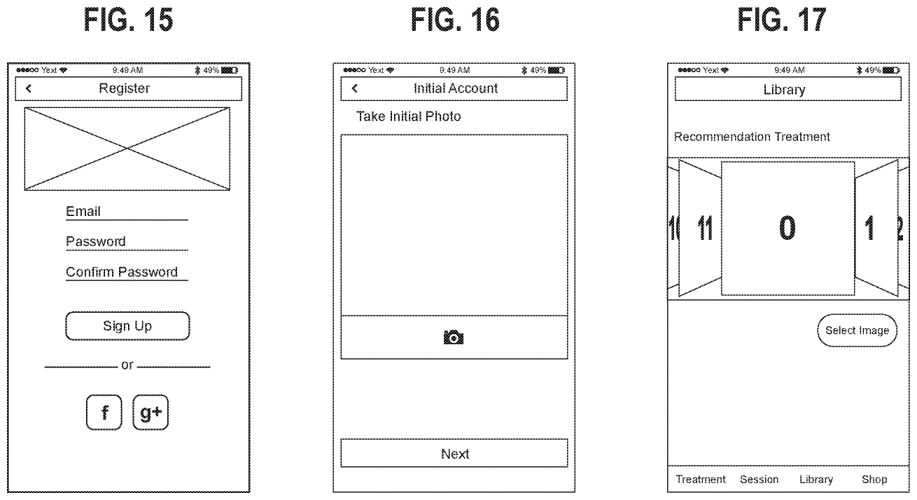

FIGS. 12A-12E are provided by way of example only, and not by way of limitation (see, e.g., the discussion of FIGS. 15-23). It will be appreciated that the user interface screens 1210 and 1230-1250 may be customized as a function of the overall operability of the software application program allowing control of panels 111, or otherwise as desired or necessitated by the features sought to be provided by apparatus 110.

In that regard, FIG. 13 is a simplified block diagram illustrating functional blocks of an illumination apparatus 110 in accordance with one embodiment. As noted above, apparatus 110 generally comprises housing 119 to support one or more illumination panels 111 and various other components; in the FIG. 13 embodiment, these other components include a power functional block 1301, regimen control software 1302, a memory component 1303, a communications interface 1304, control electronics 1305, and a sensor 1306.

Power functional block 1301 may be implemented as an internal battery (such as a Li-Ion or NiCad power cell), for instance. Additionally or alternatively, power functional block 1301 may have a suitable port to receive alternating current (AC) or direct current (DC) operating power from an external source such as an AC power source or from device 190 as noted above; in some implementations, a suitable AC/DC converter may be appropriate, depending upon the hardware configuration of power functional block 1301. In one generally available commercial implementation, power functional block 1301 may be embodied in or comprise a calendrical 18650 Li-Ion battery cell, though other types of portable power sources may also be suitable. In some implementations (such as in a “smart battery” pack embodiment), power functional block 1301 may comprise control electronics to facilitate, regulate, or otherwise to control charging and discharging cycles. It will be appreciated that power functional block 1301 may be incorporated into or combined with communications interface 1304 described below.

Regimen control software 1302 may operate independently, for example, or in cooperation with software, protocol stacks, instruction sets, function calls, and the like received from an external source (such as device 190, for example) via communications interface 1304. In operation, regimen control software 1302 provides user interface functionality and dynamic control of panel 111 via control electronics functional block 1305. In particular, regimen control software 1302 may cooperate with memory 1303 (such as for access to preprogrammed, historical, or other short term data) and control electronics 1305 to cause panel 111 to emit light in accordance with a particular predetermined or dynamically adjusted sequence of intensities and durations.

Control electronics 1305 may generally be embodied in or comprise a digital computing hardware component such as a microprocessor or microcontroller, an application specific integrated circuit (ASIC), or some other digital processor component suitable to interact with other components of system 100 to provide the functionality set forth herein. Those of skill in the art will appreciate that, in addition to those elements noted above, field programmable gate arrays (FPGAs), programmable logic controllers (PLCs), programmable single electron transistor (SET) logic components, or combinations of these and other electronic devices or components may be implemented at control electronics 1305 and suitably configured to provide some or all of the functionality necessary to drive panels 111 in cooperation with regimen control software 1302. In one generally available commercial implementation, control electronics 1305 may be embodied in or comprise a 16-bit microcontroller (for example, part number P/N PIC24FJ64GA704 available from Microchip™ Technology Inc.), though an ordinarily skilled artisan will appreciate that various alternatives are readily available from numerous manufacturers and may be suitable (or easily modified to be suitable) to support the functionality set forth herein.

In some implementations, communications interface 1304 may incorporate or be embodied in a hard-wired interface such as a USB or Ethernet port. As noted above, some communications interface hardware architectures may also provide operational power. Additionally or alternatively, suitable hardware may be employed at communications interface 1304 to enable or to facilitate wireless communications protocols such as WiFi or those employing Bluetooth or other NFC technologies. In any event, those of skill in the art will appreciate that communications interface 1304 may provide bi-directional data communications between apparatus 110 and a suitably configured external system (such as device 190) via any of various communications protocols that are generally known or that may be developed in accordance with known principals. Accordingly, control electronics 1305, memory 1303, and regimen control software 1302 resident at or incorporated into apparatus 110 may operate in cooperation with device 190 (or operate independently) to drive functionality of panel 111, to exchange regimen data stored in memory 1303, or both.

Sensor 1306 may be an infrared, ultraviolet, visual, or other electromagnetic intensity or temperature detector operative to sense conditions at the skin of the person using system 100 (or the temperature of a mask applied to the skin, for instance). Data detected by sensor 1306 may be used, for example, by control electronics 1305, regimen control software 1302, or both, dynamically to adjust wavelength, intensity, or duration of light output by panel 111. The present disclosure is not intended to be limited by any particular detection technology implemented at sensor 1306. In operation, by way of example, in the event that sensor 1306 detects a color, temperature, or other sensed characteristic at a mask or at illuminated skin that is out of range or beyond a predetermined threshold for a particular regimen or treatment protocol, then output of panel 111 may be suitably adjusted to bring the temperature or other characteristic back within an acceptable or appropriate range. Such an embodiment may have particular utility in situations where a serum or enzymatic extract used in connection with the mask is especially sensitive to temperature, where ambient atmospheric pressure or humidity affect the photosensitive nature of the serum or extract, or where a user is positioned too close to panel 111 for a specific serum or extract chemistry.

In some implementations, regimen control software 1302 may include a manual setting functional block (not shown) that enables a user to customize a particular treatment protocol. In cooperation with memory 1303, control electronics 1305, and regimen control software 1302 operating in accordance with the aforementioned software application, such a manual setting functional block may directly affect output of panel 111 in a manner specified via user interface operative elements discussed above with reference to FIGS. 12A-12E. Accordingly, device 110 may be programmed effectively to manipulate output (wavelength, intensity, and duration) of panel 111 as desired; in some situations, it may be useful to store such a manually created treatment protocol or sequence in memory 1303 for subsequent retrieval, use, or modification, and a user interface to facilitate saving and retrieving such protocols may be readily be implemented in regimen control software 1302.

FIG. 14 is a simplified flow diagram illustrating general operational flow of one embodiment of a method in accordance with the present disclosure. In the illustrated method, user of a software application program may be authenticated as indicated at block 1401. As noted above, such a software application may be executed on an external device such as device 190 which is operably coupled to apparatus 110; alternatively, the application program may execute on apparatus 110, in whole or in part. Authenticating a user such as at block 1401 may involve, for instance, receiving a user name and password; where device 190 or apparatus 110 are appropriately configured, user authentication may include fingerprint identification or comparing other biometric data with information stored, for instance, in memory 1303 or some other memory store associated with or accessible by device 190. In operation, user authentication enables system 100 to acknowledge a particular user and to access and retrieve user-specific data (such as from memory 1303, for instance). These data may include a user's age and skin care requirements, preferred or prescribed treatment regimens, historical usage data, medicines and/or extracts that are typically used or contra-indicated, light wavelengths and intensities that are readily tolerated or are identified as irritable, and other relevant skin care parameters. For example, comparison of historical data may enable an application program to recommend certain treatments based upon past use or an interim since a prior or previous regimen; alternatively, an application program suitably provided with comprehensive historical data may forbid use or application of a particular regimen to prevent a user from over-treating a skin condition or potentially damaging an area of skin to which extracts and illumination are provided in accordance with a selected protocol. It will be appreciated that providing a software application program with user-specific data may be desirable in various circumstances to effectuate any number of results, and so the type and quantity of such data, and the duration that it is maintained in memory 1303, may vary in accordance with expected or desired use of system 100.

Regimen parameters may be received as indicated at block 1402. As noted above, such parameters may be received by regimen control software 1302 that is operative, in cooperation with control electronics 1305, to drive panels 111 to provide illumination. In some embodiments, the regimen parameters may be input using the user interface mechanisms described above, and may involve interaction with display 191 associated with device 190. For example, regimen parameters may be selected in accordance with one or more options provided by mode selection screen 1230, or “canned” or predetermined regimen parameters may be provided by selecting, by way of example, a saved regimen from a library or a relax button 1224 that is operative to cause the software application to execute a pre-programmed relaxation regimen. It is noted that regimen parameters may be influenced by or selected in accordance with a particular serum or enzymatic extract disposed on the skin (or on a mask applied to the skin) which is to be illuminated. Accordingly, the user interface may allow a user to identify a particular such serum or extract, and the application program may select appropriate light wavelengths, intensity settings, and durations for the regimen as a function of the light-activated chemistries of the constituents of the serum or extract. In any event, the application program may then provide such regimen parameters to regimen control software 1302, control electronics 1305, or both.

Received regimen parameters may be compared to historical data as indicated at block 1403. As noted above, it may be useful to compare a desired treatment to historical data and other information in order to determine whether a particular treatment is being over-used, or possibly even contra-indicated, based upon recent or long-term operation by a specific user. Where a treatment is not recommended or contra-indicated, the method may cease, or a warning may be displayed to a user, who would then need to override such an interrupt to continue configuring or initiating the desired regimen (this interrupt procedure is not illustrated in FIG. 14). Where historical or other user data suggest that a selected or desired regimen is appropriate or otherwise not disallowed, the method may continue to block 1404.

Illumination may be dynamically adjusted as indicated at block 1404. In that regard, panels 111 may be driven to provide illumination substantially as set forth above. Various combinations of light wavelengths, intensities, and pulse or fade durations may be applied as noted above with reference to FIGS. 9 and 10, and numerous other strategies may be appropriate, depending upon user preference, serum or extract photodynamic properties, historical data, or a combination of these and other factors. Specifically, block 1404 and the decision block illustrated at 1405 represent application or execution of the treatment regimen for a particular session. The application software may periodically poll regimen control software 1302 or memory 1303, for example, to determine if the regimen is complete; additionally or alternative, the regimen itself may be configured, for instance, under software control 1302, to announce completion to the application program. As another alternative, a simple timer may be employed at decision block 1405 to determine completion of a particular treatment regimen. If the regimen is not complete, the method may loop back to block 1403 to access additional historical data or other information that may be relevant to the current session, and an appropriate interrupt may be generated if warranted (e.g., based on duration of illumination, current color or temperature of the treated skin or a mask applied to the treated skin (as measured by sensor 1306), or other factors).

Where it is determined (at block 1405) that the treatment regimen or session has been completed, the method ends at block 1499. Data associated with the completed session may be stored, for instance in memory 1303, memory associated with device 190, or both. Such data may include a time and date for the regimen as well as other regimen parameters such as intensity, duration, identification of sera or extracts employed, whether the regimen as a whole or certain regimen parameters were prescribed by a physician or other care-taker, and the like. These stored data may become part of the historical record and data repository that are accessible by the application program as set forth above.

It is noted that the functionality depicted in the flow diagram of FIG. 14 may be effectuated in a different order, or functional blocks may be rearranged or omitted entirely, for example, as a function of the chemistry of the serum or extract on a mask (or whether a mask is even used), the duration or intensity of the emissions from panels 111, historical or prescribed treatment regimens, or a combination of these and other factors. The present disclosure is not intended to be limited by the order or arrangement of the blocks illustrated in FIG. 14.

FIGS. 15-23 illustrate different user interface screens facilitating interaction with a system in accordance with some embodiments. FIGS. 15 and 16 illustrate a registration screen and an initial account screen, respectively. As indicated in FIG. 15, a user may be prompted to create an account (i.e., register or sign in) such that a user-specific data storage area may be created to maintain person preferences, historic data, recommendations, disallowed or disfavored treatments, and the like. In that regard, a user may be prompted to enter a username or e-mail address (for identification purposes) and a password (for security purposes); as noted above, fingerprint identification or other biometric data may replace or supplement a password. As indicated in FIG. 16, a user may be prompted to enter a photograph to be associated with the created user profile and stored in the user-specific data storage area. In some embodiments, the photograph may be used as part of the biometric data for user identification or authorization (as described above with reference to FIG. 14), for example, to allow a user to sign in and to access profile or other user-specific data and preferences. Additionally or alternatively, such a photograph may serve as a “baseline” image representative of a skin condition at a particular moment in time, e.g., prior to commencement of a long-term treatment schedule; in these circumstances, subsequent image captures of the same region of the skin may be compared to the initial image that is prompted in FIG. 16, for example, to monitor or otherwise to measure changes or progress that may be attributable to ongoing treatments.

FIG. 17 illustrates one embodiment of a library screen. As illustrated, a user may scroll through a list of twelve recommended or frequently used treatments or regimens, though any number of such treatments may be stored in a library (e.g., in memory 1303) for subsequent access and retrieval. As depicted, the user interface presents a “jukebox style” representation of the items stored in the library, but any number of other paradigms are equally useful and may be employed as a design choice. For example, a grid of icons may be used with each icon representing a different treatment, or a scrollable list of treatment names may be provided in text format. Each treatment stored in the library may have all or a selected subset of required regimen parameters associated therewith, such that selection of a particular treatment from the library screen allows the software application program to access necessary parameters to instantiate (and complete) the regimen.

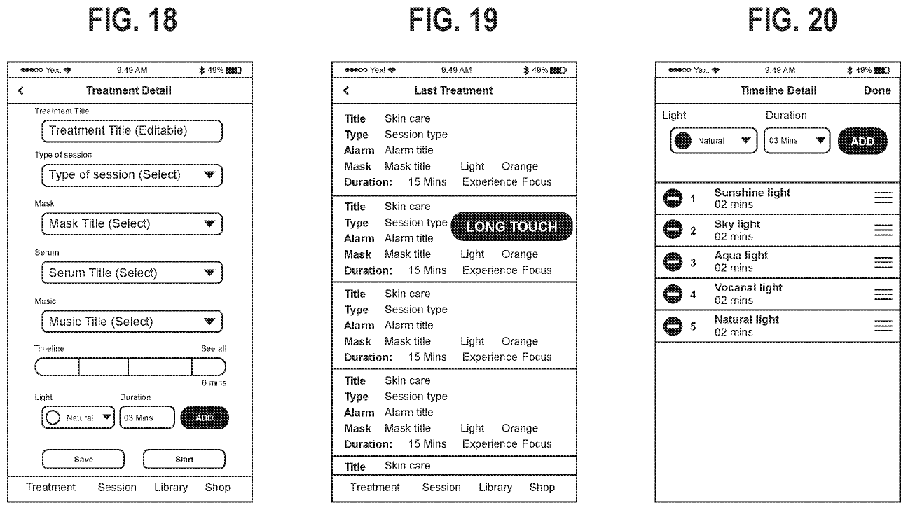

FIG. 18 illustrates one embodiment of a treatment detail screen which facilitates entry of specific treatment parameters; these parameters may be saved, as indicated near the bottom of FIG. 18, such as in a library or other data store substantially as set forth above. In the FIG. 18 embodiment, the desired treatment may be provided a name to facilitate identification and subsequent retrieval and re-use. Additional parameters may include some or all of the following: characterization of the type of treatment (e.g., acne treatment, post-surgical recuperative treatment, moisturizing treatment, relaxation treatment, and the like); whether and what type of mask, serum, or extract is used in association with the treatment; illumination types, intensities, and durations to be delivered by panels 111; and audio or visual preferences. As noted above, other parameters may be relevant for a particular treatment, and so the user interface illustrated in FIG. 18 may be altered to accommodate the type of treatments contemplated by system 100 or the software application program executing thereon.

FIG. 19 illustrates one embodiment of a previous treatment overview screen; as illustrated, the overview screen identifies four previous treatments by name, duration, and other relevant parameters, though other embodiments may display more or fewer than four previous treatments. A user interface mechanism may enable a user to select a particular previous treatment for further analysis. In the illustrated embodiment, a “long touch” (i.e., tap and hold) input may be used to select a particular displayed treatment, but double tapping or other user interface paradigms may also be used. Upon selection of a particular previous treatment, a user may be presented with a display of additional detail and treatment parameters via a different screen, such as that depicted in FIG. 18, for example.

FIG. 20 illustrates one embodiment of a timeline detail screen associated with a particular treatment. The timeline detail may provide more information than the timeline identified in FIG. 18, for instance, and may allow deletion or alteration of particular time segments associated with a particular treatment regimen.

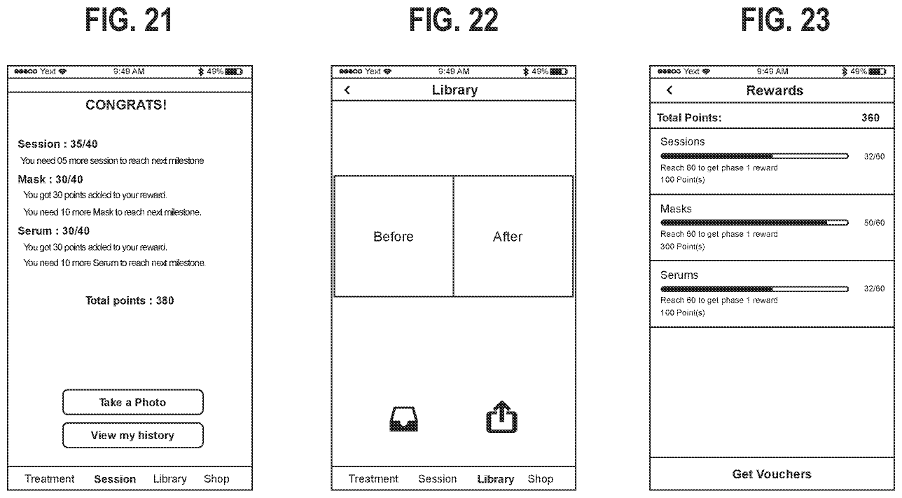

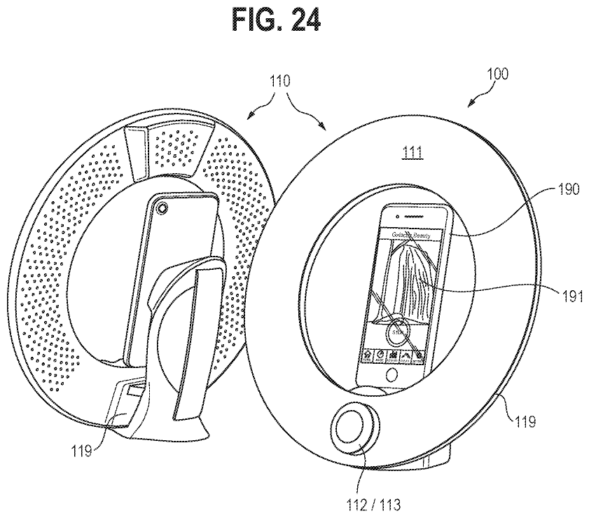

FIGS. 21 and 23 illustrate embodiments of a session complete screen and a rewards progress screen, respectively. Upon completion of a particular session or treatment regimen, the FIG. 21 embodiment may indicate completion of the session and points or other rewards indicia associated with the particular treatment regimen just completed; the rewards progress screen of FIG. 23, in contrast, may indicate overall progress towards a particular rewards goal, and may not be limited to data associated with (or reward points accrued as a result of) completion of any particular treatment regimen. In the FIG. 23 embodiment, for instance, a voucher for free or discounted masks or sera may be obtained upon accumulation of sufficient points, which may be associated with past and/or continuing use of the same or similar consumable products. It is noted that embodiments contemplating rewards programs or discounts offered in exchange for use of system 100 are entirely optional. In particular, the disclosed subject matter has utility as set forth above independent of such rewards or discount programs, however, these programs can have utility in inspiring loyalty and incentivizing continued use of system 100 in many situations.

FIG. 22 illustrates one embodiment of a “before and after” screen facilitating comparison of an area of the skin prior to treatment, on the one hand, with the same area of the skin following treatment, on the other hand. As noted above with reference to FIG. 16, a baseline image representative of a skin condition prior to commencement of a long-term treatment schedule may be compared to a similar image of the same area acquired post-treatment to facilitate monitoring or measuring progress, efficacy of the treatment regimen, and the like. In the FIG. 22 embodiment, these before and after photographs may be presented to a user for visual inspection to allow the user to evaluate the effects of treatments over time; additionally or alternatively, the software application program itself may employ image processing techniques, for example, to monitor changes in various aspects or visual characteristics of areas of the skin over time. In such embodiments, the software application program may be enabled to make a determination regarding treatment efficacy or efficiency, which may drive recommendations for future treatment regimens or protocols, session frequencies, and the like.

It is noted that the screen shots depicted in FIGS. 12 and 15-23 are provided by way of example only, and not by way of limitation. Many operational flows may be implemented depending upon, for instance, user preference, treatment history, the operational characteristics or limitations of panels 111, or care provider recommendations; accordingly, user interface paradigms and operative interface elements may vary as a design choice based upon these or a variety of other considerations generally known in the art or developed in accordance with known principals.

### Overview and Use Cases

As set forth above, system 100 may provide a home or portable treatment apparatus that is engineered to utilize multiple wavelengths of light (such as from an LED panel or other suitable panel technology) and wireless (such as WiFi, Bluetooth, or other NFC) technology to interact with an individual's device (such as a smartphone or tablet) to personalize a skincare treatment using innovative, light activated ingredients in skincare products for the face, neck, and chest skin. The various wavelengths may be selected to target specific skin concerns such as acne, rosacea, redness, sun damage, fine lines, wrinkles, large pores, and dryness. Targeted LED (or other) lighting wavelengths may also promote better sleep patterns for those who suffer from insomnia and stress.

Skin concerns such as acne, rosacea, redness, sun damage, fine lines, wrinkles, large pores, and dryness are problems that affect millions of people on a chronic basis. These problems are worsened by lack of sleep which affects 30% of all adults. Acne affects 85% of people ages 18-25, and is the most wide-spread skin condition in the United States, affecting an estimated 50 million people a year. (See, e.g.: Bhate K, Williams H O. Epidemiology of acne vulgaris. The British journal of dermatology 2013:168:474-85; and Bickers D R, Urn H W, Margolis D, Weinstock M A, Goodman C, Faulkner E et al. The burden of skin diseases: 2004 a joint project of the American Academy of Dermatology Association and the Society for investigative Dermatology. Journal of the American Academy of Dermatology 2006; 55:490-500.) Rosacea affects nearly 16 million adults in the United States and is increasing due to the increased exposure to chemical and environmental pollutants. Meanwhile, the treatment for many of these problems is optimized by laser technologies which are expensive and inaccessible to many people who cannot afford to pay for them in a doctor's office or medical spa.

System 100, however, may be implemented in such a way that it is affordable, accessible, and adaptable to many skin problems and to different skin types. Accordingly, system 100 may provide a solution for millions of youth and adults worldwide. The distinct wavelengths emitted by panels 111 (which may implement LED or other emissive technologies) are healing for specific issues with the skin, and will further improve any treatment regimens recommended by a physician or care-giver.

System 100 is the first in class to utilize emissive technology in communication with a software application program that may personalize each treatment regimen and vary the combination of different wavelengths for each treatment depending on the skin type, skin condition, age, gender, and progress within a treatment strategy or protocol. System 100 is the first solution to activate photodynamic (and “over the counter” or OTC) ingredients in cosmeceuticals to maximize targeted treatment of specific skin conditions, and it is the first photodynamic solution to embrace the connection between good sleeping patterns and skin health by employing science-based treatment for inducing better sleep.

It is to be appreciated that the foregoing Detailed Description section, and not the Abstract section, is intended to be used to interpret the claims. The Abstract section may set forth one or more, but not all, examples of the subject matter described in the present disclosure, and thus, is not intended to limit the present invention and the appended claims in any way.

The foregoing description of the specific embodiments should fully reveal the general nature of the disclosure so that others can, by applying knowledge within the skill of the art, readily modify and/or adapt for various applications such specific embodiments, without undue experimentation, without departing from the general concept of the present disclosure. Since many modifications, variations and changes in detail can be made to the described subject matter, it is intended that all matters in the foregoing description be interpreted as illustrative and not in a limiting sense. Thus, the scope of the invention should be determined by the appended claims and their legal equivalents. Moreover, the breadth and scope of the present disclosure should not be limited by any of the above-described examples, but should similarly be defined only in accordance with the following claims and their equivalents.

All patents, patent applications, provisional applications, and publications referred to or cited herein are incorporated by reference in their entirety, including all figures and tables, to the extent they are not inconsistent with the explicit teachings of this specification. By citation of various references in this document, Applicants do not admit any particular reference is “prior art” with respect to the subject matter described and claimed herein.

What is claimed is:

1. A system of photodynamic skin therapy, the system comprising:
an apparatus comprising a housing operative to support a light emissive panel;
a user interface operative to receive parameters associated with the skin therapy;
regimen control software operative to provide instructions related to the parameters; and
control electronics operative to cause said light emissive panel to emit light of a selected wavelength in accordance with the instructions.

2. The system of claim 1 wherein said apparatus further comprises a memory storing data associated with the skin therapy and wherein said regimen control software accesses the data to provide the instructions.

3. The system of claim 1 wherein said apparatus further comprises a communications interface and wherein said regimen control software receives the parameters via said communications interface.

4. The system of claim 3 further comprising a remote device coupled to said apparatus via said communications interface, wherein said remote device is operative to execute an application program to provide the parameters to said regimen control software.

5. The system of claim 4 wherein said remote device is a wireless telephone.

6. The system of claim 5 wherein said user interface allows interaction with the application program executing on the wireless telephone.

7. The system of claim 1 wherein the light has a wavelength from about 420 nm to about 460 nm.

8. The system of claim 1 wherein the light has a wavelength from about 630 nm to about 700 nm.

9. The system of claim 1 wherein the light is operative to activate a photodynamic compound.

10. The system of claim 1 wherein said apparatus further comprises a sensor operative to obtain effect data from an area of skin illuminated by the light.

11. A system of photodynamic skin therapy, the system comprising:
a hydrogel matrix for application to an area of skin;
a topical composition comprising an enzymatic extract embedded within said hydrogel matrix;
an apparatus comprising a housing operative to support a light emissive panel;
a user interface operative to receive parameters associated with the skin therapy;
regimen control software operative to provide instructions related to the parameters; and
control electronics operative to cause said light emissive panel to emit light of a selected wavelength in accordance with the instructions;
wherein a portion of said hydrogel matrix is selectively illuminated by the light.

12. The system of claim 11 wherein said apparatus further comprises a sensor operative to obtain effect data from an area of said hydrogel matrix illuminated by the light.

13. The system of claim 12 wherein said regimen control software selectively modifies the instructions responsive to the effect data.

14. The system of claim 11 wherein said topical composition further comprises at least one of Baobab seed extract, wild indigo (Tephrosia purpurea) extract, pre- and/or pro-biotic extracts, and a carrier.

15. The system of claim 11 wherein the enzymatic extract is a photodynamic compound.

16. The system of claim 11 wherein the enzymatic extract is a thermodynamic compound.

17. A method of photodynamic skin therapy, the method comprising:
embedding a topical composition within a hydrogel matrix, the topical composition comprising an enzymatic extract;
applying the hydrogel matrix to an area of skin; and
selectively illuminating a portion of the hydrogel matrix in accordance with a skin treatment regimen and the topical composition;
wherein said selectively illuminating comprises:
providing an apparatus comprising a housing operative to support a light emissive panel;
receiving parameters associated with the skin treatment regimen via a user interface;
instantiating regimen control software operative to provide instructions related to the parameters; and
employing control electronics operative to cause the light emissive panel to emit light of a selected wavelength in accordance with the instructions.

18. The method of claim 17 wherein said selectively illuminating further comprises employing a sensor operative to obtain effect data from the portion of the hydrogel matrix and selectively modifying the instructions responsive to the effect data.

19. The method of claim 17 wherein the topical composition further comprises at least one of Baobab seed extract, wild indigo (Tephrosia purpurea) extract, pre- and/or pro-biotic extracts, and a carrier.

20. The method of claim 17 wherein the enzymatic extract is a thermodynamic compound.
